(19) United States
(12) Patent Application Publication
(10) Pub. No.: US 2026/0284124 A1
(43) Pub. Date: Sep. 24, 2026

(54) FOOD SUPPLEMENT CONTAINING THE COMBINATION OF SINGLE KIND AND HIGH DOSE PROBIOTICS IN ONE PLACE

(71) Applicant: BIOPROBIF SAGLIK GIDA ARGE SANAYI VE TICARET LIMITED SIRKETI, Istanbul (TR)

(72) Inventor: Hüseyin Sancar BOZKURT, Istanbul (TR)

(73) Assignee: BIOPROBIF SAGLIK GIDA ARGE SANAYI VE TICARET LIMITED SIRKETI, Istanbul (TR)

(21) Appl. No.: 18/996,195

(22) PCT Filed: Aug. 29, 2022
(86) PCT No.: PCT/TR2022/050918

§ 371 Date: Jan. 17, 2025

(30) Foreign Application Priority Data
Aug. 24, 2022 (TR) 2022/013347

(51) Int. Cl. A61K 35/745; A61K 9/00; A61K 35/00; A61K 47/26; A61K 47/36

(52) U.S. Cl. CPC A61K 35/745; A61K 9/0095; A61K 47/26; A61K 47/36; A61K 2035/115

(57) ABSTRACT

A food supplement containing a combination of single kind and high dose probiotics having natural ingredients in one place, which strengthens immunity, has the property to destroy bacteria, viruses, and microplastics, and regulates the intestinal microbiota.

### TECHNICAL FIELD

The invention relates to a food supplement containing the combination of prebiotic, probiotic bacteria and postbiotic products in one place which is used as a food supplement that protects human health and helps treatment, which is suitable for oral and other use, which is immune-enhancing and which has the properties to destroy microplastics in the intestine.

### BACKGROUND

Today, many people need to use one or more vitamins or food supplements due to intense work schedule or other conditions. Minerals, medicinal herbs, botanicals, amino acids and enzymes are used in the production of food supplement types used in addition to vitamins. However, food supplements are offered to the users in the form of tablets, capsules, chewing gum, powder, beverages and energy bars. Food supplement can help to strengthen the immune system against harmful microorganisms.

These products, whose main function is to provide the nutrients to the body that cannot be consumed in sufficient quantities are the nutrients presented in the forms of vitamins, minerals, amino acids, fatty acids, pills, tablets, capsules, liquids, etc. The food supplements are prepared in various forms by using nutrients such as vitamins, minerals, proteins, amino acids, herbs, enzymes, fiber and fatty acids. These food supplements do not aim to treat or prevent any disease. In general, the food supplements do not replace natural nutrition, but it has the property to complement its deficiencies.

The food supplements can meet the daily needs of people or they are used to support their current treatment.

The food supplements with probiotics used in the current situation can strengthen the body protection and maintain the balance of beneficial bacteria in the gut, and helps to increase the antibody production and to reduce the risk of COVID-19 symptoms.

However, the use of more than one bacteria in this food supplement used may cause confusion in terms of the effect mechanisms of the bacteria. As a prebiotic, there is a nutrient that harmful bacteria can also use.

In the invention, a single type of probiotic bacteria always has a beneficial effect, no matter how high the dose is. Only Bifidobacteria probiotic bacteria is used as the XOS prebiotic contained in the invention. In addition, scientifically proven alginic acid is used in the content.

This invention is a supplementary food containing the combination of Prebiotic-Probiotic-Postbiotic supplements which has an immune-enhancing effect and which has the property to destroy the microplastics harmful to the intestines and the environment. It contains booster and Bifidobacterium Infantis probiotic. Xyloglucan, a xylooligosaccharide used as a prebiotic, has the property to increase the healing effect of Bifido bacteria in inflammatory bowel diseases.

It is a food supplement that helps in the treatment of various diseases on humans, especially for problems such as bacteria, viruses, inflammation. It is suitable for use in all age ranges and pregnant women.

The structural and characteristic features of the invention and all of its advantages will be understood more clearly thanks to the detailed description written below, and therefore this evaluation should be made by taking these figures and detailed description into consideration.

### DETAILED DESCRIPTION OF THE EMBODIMENTS

The invention, the food supplement containing the combination of single kind and high dose probiotics in one place, is described in the following detailed description without any limiting effect.

The materials and weight ratios required for the food supplement containing the combination of single kind and high dose probiotics in one place which is the subject of the invention are as follows:
Bifidobacterium Infantis: 10 -500 billions,
Xylooligosaccharide: 15-80 grams,
Alginic acid: 3-20 grams.

In the invention, minimum: 10 billions of Bifidobacterium Infantis, 15 grams of xyloglucan, 3 grams of Alginic acid and maximum: 500 billions of Bifidobacterium Infantis, 80 grams of xyloglucan, 20 grams of Alginic acid are used in the range.

Bifidobacterium Infantis bacteria, which is used as a probiotic bacteria, affects the intestines by regulating immunity and by digesting and destroying the microplastics accumulated in the intestine by means of mixing with water and drinking in a way to be numerically 10-500 billions of colonies as CFU/ml in powder form or by means of endoscopic application.

Xyloglucan (Xylooligosaccharide), which is a plant-derived hemicellulose used to feed Bifidobacterium Infantis probiotic bacteria, increases the number and effect of Bifidobacteria in the intestines it reaches by mixing and drinking it with water in 15-80 grams of powder form or by endoscopic application.

Alginic acid (Alginate), which is obtained from the cell membrane of brown seaweed used as a postbiotic in the invention, provides regulation in the intestines where it reaches by mixing and drinking it with water in 3-20 grams of powder form or by endoscopic application. It has a protective effect.

Bifidobacterium Infantis used in the invention has the property to enhance the immunity and to destroy the microplastics. Bifidobacterium infantis is a probiotic commensal heterotrophic anaerobic bacterium that regulates the intestinal microbiota. There is probiotic bifidobacterial biodegradation of microplastics, which is a global environmental problem that threatens human health. Supportive single strain probiotic bifidobacteria are effective in treatment for mild/moderate/severe SARS-CoV-2 patients.

Xylooligosaccharide has been added to increase the number and effect of Bifidobacteria. Xyloglucan, a plant-based prebiotic oligosaccharide, causes an increase in the amount of bifidobacteria.

Alginic acid contributes to the creation of the environment in which Bifidobacteria can reproduce. The crystal chains in the alginic acid structure are split by bacteria.

The preparation and process steps of the food supplement which is the said invention are as follows:
The mixture obtained by mixing the 3 components currently available in powder form at room temperature, in a dry environment and mixing them all at the same time, can be stored by being filled in sachet and/or capsule form at room temperature without opening the package or without contacting with water after opening.

The combination of Bifidobacterium Infantis bacteria, xylooligosaccharide (XOS) (xyloglucan) prebiotic and Alginate (alginic acid and sodium salts) ingredients in one place is provided. The said invention provides the use of vitamins and minerals by the body in humans.

In addition to oral use, the invention can be applied endoscopically and colonoscopically. This invention provides benefit naturally without any additive content.

It is clear that a person skilled in the art can demonstrate the innovation revealed in the invention by using similar embodiments and/or can apply this embodiment to other similar purposes used in the related art. Therefore, it is obvious that such embodiment will lack the criteria of innovation and especially going beyond the state of the art.

1-5. (canceled)

6. A food supplement containing the combination of single kind and high dose probiotics, comprising:
Bifidobacterium Infantis: 10 -500 billions of colonies,
Xylooligosaccharide: 15-80 grams, and
Alginic acid: 3-20 grams.

7. The food supplement containing the combination of single kind and high dose probiotics according to claim 6, wherein the Bifidobacterium Infantis is used for drinking the probiotic bacteria by means of mixing with water and drinking in a way to be numerically 10-500 billions of colonies as CFU/ml in powder form or by endoscopic application.

8. The food supplement containing the combination of single kind and high dose probiotics according to claim 6, wherein the Xylooligosaccharide is a plant-derived hemicellulose used to feed Bifidobacterium Infantis probiotic bacteria by drinking it by mixing with water in 15-80 grams of powder form mixture or by endoscopic application.

9. The food supplement containing the combination of single kind and high dose probiotics according to claim 6, wherein the alginic acid is obtained from a cell membrane of brown seaweed and is used as a postbiotic by mixing and drinking it with water in 3-20 grams of powder form mixture or by endoscopic application.

10. The food supplement containing the combination of single kind and high dose probiotics according to claim 6, wherein a mixture is obtained by mixing the Bifidobacterium Infantis, Xylooligosaccharide, and alginic acid in powder form at room temperature, in a dry environment, and mixing them all at the same time, and is then filled in sachet and/or capsule form.
